(19) United States
(12) Patent Application Publication
(10) Pub. No.: US 2019/0343861 A1
(43) Pub. Date: Nov. 14, 2019

(54) METHOD OF USING PROTEIN KINASE A ACTIVATOR AND INHIBITOR IN PREPARATION OF DRUGS FOR TREATING DISEASES ASSOCIATED WITH CHANGES IN PLATELET COUNTS AND FOR INHIBITING AND PROMOTING PLATELET APOPTOSIS

(71) Applicant: Soochow university, Jiangsu (CN)

(72) Inventor: Kesheng DAI, Jiangsu (CN)

(73) Assignee: Soochow university, Jiangsu (CN)

(21) Appl. No.: 16/520,372

(22) Filed: Jul. 24, 2019

Related U.S. Application Data
(63) Continuation-in-part of application No. PCT/CN2017/112898, filed on Nov. 24, 2017.

(30) Foreign Application Priority Data
Jan. 25, 2017 (CN) 201710060730.4
Jan. 25, 2017 (CN) 201710060759.2

(51) Int. Cl. A61K 31/7076

(52) U.S. Cl. CPC A61K 31/7076

(57) ABSTRACT

A method of using protein kinase A (PKA) activator and inhibitor in preparations of drugs for treating diseases associated with changes in platelet counts. Protein kinase A regulates platelet apoptosis by means of regulating the phosphorylation of serine at BAD position 155; the activation of protein kinase A activity may inhibit the occurrence of endogenous platelet apoptosis, and may also increase circulating platelet counts in experimental animals; in addition, inhibiting PKA activity may induce platelet apoptosis in vitro, while also reducing circulating platelet counts in the body, indicating that PKA inhibitors may participate in the treatment of thrombocytosis, and reduce platelet counts in peripheral circulating blood.

### CROSS-REFERENCE TO RELATED APPLICATION

This application is a continuation-in-part of international PCT application serial. no. PCT/CN2017/112898, filed on Nov. 24, 2017, which claims the priority benefit of China application no. 201710060730.4, filed on Jan. 25, 2017, and Chinese application serial no. 201710060759.2, filed on Jan. 25, 2017. The entirety of each of the above-mentioned patent applications is hereby incorporated by reference herein and made a part of this specification.

### FIELD OF THE INVENTION

The present invention belongs to the field of platelet related drugs, and particularly relates to a method of using the protein kinase A activator and inhibitor in preparation of drugs for treating diseases associated with changes in platelet counts.

### DESCRIPTION OF RELATED ART

Platelets finely regulate thrombosis and the balance of hemorrhage associated with blood circulation. At the same time, platelets play important roles in many important pathophysiological processes, such as immunity, infection, arteriosclerosis, tumor development and metastasis, and the like. However, the lifespan of platelets is short and mysterious. Why platelets only circulate in the body for 8 to 9 days? This question has puzzled humans for more than half a century. In addition, life-threatening thrombocytopenia usually occurs in many high-incidence diseases such as diabetes, infection, and ITP, and after many pharmacological treatments. The reasons for shortening of the platelet lifespan in these pathological processes are not fully understood. Self-limitation, short lifespan, and especially pathological changes during storage limit the effective period of stored platelets for treating thrombocytopenia. Therefore, finding a mechanism to regulate the platelet lifespan and survival has important pathophysiological significance.

Platelets are a key factor in regulating thrombosis and pathological hemorrhage in the circulatory system, and also play an important role in the pathophysiological processes such as body's immune response, infection, atherosclerosis, and tumor metastasis. Fine regulation of the life cycle of platelets is the key to maintain the platelet counts in normal humans. Increase in platelet counts is commonly seen in many diseases, such as essential thrombocytosis and polycythemia vera. In some pathological processes, the increase of platelet counts in peripheral blood increases the risk of hemorrhage or thrombosis, such as chronic myelocytic leukemia, post-massive hemorrhage, chronic inflammation and tumors. Therefore, exploring the mechanism to regulate the platelet lifespan and survival, and reducing the platelet counts in peripheral blood by shortening the platelet lifespan have an important pathophysiological significance for the treatment of thrombocytosis.

In recent years, more and more attention has been paid to research on platelet apoptosis because it can reveal the mystery of the platelet lifespan and survival. There is increasing evidence that intrinsic procedures of cell apoptosis under pathological and physiological conditions lead to platelet destruction. Similar to eukaryotic cells, BAK and BAX that are two destined killers in the cell do not deplete during platelet involved thrombosis and hemostasis. However, among many anti-apoptotic proteins of the Bcl-2 family, it has been demonstrated that only Bcl-xL and BAK are involved in the regulation of apoptosis of anucleate platelets. Mutant Bcl-xL dose-dependently reduces platelet survival in vivo, and this process can be inhibited by knocking out BAK and BAX. P53 has been proven to be involved in the regulation of platelet apoptosis by inhibiting Bcl-xL activity. In addition, BAD, namely the anti-apoptotic protein Bcl-2 homeodomain 3 (BH3) protein, is also found to be involved in the regulation of platelet survival. Knocking out BAD can significantly extend the platelet lifespan. These studies have revealed a key role of platelet apoptosis proteins in regulating the platelet lifespan. However, the basic problem remains, and how to induce or inhibit platelet apoptosis under physiological or pathological conditions is still unclear currently.

Protein kinase A (PKA) is a serine-threonine protein kinase that is widely present in eukaryotic cells. PKA is a heterotetramer composed of two catalytic subunits and two regulatory subunits. Upon binding to the regulatory subunits, cyclic adenosine monophosphate releases the activated catalytic subunits, which in turn regulate various activities in the cell, including cell metabolism, growth, differentiation, gene expression, and cell apoptosis. PKA is highly expressed in platelets, and PKA plays an important role in the regulation of platelet functions. However, whether PKA has a great impact on platelet apoptosis induced by storage or pathological stimulus remains unclear.

### SUMMARY OF THE INVENTION

### Technical Problem

Platelet apoptosis limits its lifespan, and platelet apoptosis caused by many diseases can cause thrombocytopenia, but the initiation and regulation mechanism of platelet apoptosis has not yet been fully elucidated. The technical problem we are trying to solve is to further study the specific mechanism by which a protein kinase A activator and inhibitor inhibit and promote platelet apoptosis, and further disclose the use of the protein kinase A activator and inhibitor in preparation of drugs for treating diseases associated with changes in platelet counts.

### Technical Solution

In view of the above problems, the present invention discloses a method of using a protein kinase A activator in preparation of drugs for treating diseases associated with decrease in platelet counts.

Preferably, the protein kinase A activator is one or more of an inorganic activator and an organic activator.

Preferably, the inorganic activator is one or more of hydrides, oxides, acids, bases, and salts.

Preferably, the organic activator is one or more of hydrocarbons, hydrocarbon derivatives, saccharides, proteins, fats, nucleic acids, and synthetic polymer materials.

Preferably, the hydrocarbons are one or more of alkenes, alkanes, alkynes, and aromatic hydrocarbons; the hydrocarbon derivatives are one or more of halogenated hydrocarbons, alcohols, phenols, aldehydes, acids, and esters; the saccharides are one or more of monosaccharides, disaccharides, oligosaccharides, and polysaccharides; the proteins are one or more of amino acids and polypeptides; and the nucleic acids are one or more of deoxyribonucleic acid and ribonucleic acid.

Preferably, the protein kinase A activator is one or more of a phosphodiesterase inhibitor, an adenylate cyclase agonist, a cyclic adenosine monophosphate and a substrate of protein kinase A catalytic subunit, or analogs and derivatives made according to structural features thereof.

Preferably, the protein kinase A activator is adenosine triphosphate or analogs and derivatives made according to structural features thereof.

Preferably, the protein kinase A activator is one or more of drugs amrinone, milrinone, enoximone, aminophylline, dinoprostone, iloprost, cilostazol, cilostamide, and dipyridamole or analogs and derivatives made according to structural features thereof.

Preferably, the protein kinase A activator is one or more of Ginkgo biloba extract, quercetin, meglumine adenosine cyclophosphate, cyclic adenosine monophosphate, forskolin, 8-bromoadenosine-3′,5′-cyclic monophosphate, 8-bromo-cyclic adenosine monophosphate, 8-piperidinyladenosine-cyclic adenosine monophosphate, 8-chloro-cyclic adenosine monophosphate, adenosine 3,5-cyclic monophosphate, N6-benzoyl-cyclic adenosine monophosphate, (S)-adenosine, cyclic 3′,5′-(hydrogen phosphorothioate)triethyl, 3-isobutyl-1-methylxanthine, 8-chlorophenyl-cyclic adenosine monophosphate, adenosine 3,5-cyclic monophosphate, adenosine 3,5-cyclic monophosphorothioate, 8-bromo-cyclic adenosine monophosphate, specific 5,6-4,5-dicyanoimidazole-cyclic adenosine monophosphate, specific 8-chlorophenyl-cyclic guanosine monophosphate sodium salt, specific adenosine 3′,5′-cyclic monophosphorothioate triethyl salt, specific cyclic adenosine monophosphate, dibutyryl-cyclic adenosine monophosphate, N6-monoacyladenosine 3′,5′-cyclic monophosphate, 8-bromoadenosine 3′,5′-cyclic monophosphorothioate, 8-bromoadenosine 3′,5′-cyclic monophosphate, N6-benzoyl-cyclic adenosine monophosphate, and erythro-9-amino-β-hexyl-α-methyl-9H-purine-9-ethanol hydrochloride-9-adenine hydrochloride or analogs and derivatives made according to structural features thereof.

Preferably, the diseases associated with decrease in platelet counts include immune thrombocytopenia, infection-induced thrombocytopenia, secondary thrombocytopenia, drug-induced thrombocytopenia, thrombopoiesis deficiency disease, or non-immune thrombocytopenia.

Preferably, the immune thrombocytopenia includes idiopathic thrombocytopenic purpura.

Preferably, the infection-induced thrombocytopenia includes bacterial infection-induced thrombocytopenia or viral infection-induced thrombocytopenia.

Preferably, the secondary thrombocytopenia includes thrombocytopenia in diabetic patients, thrombocytopenia in tumor patients, thrombocytopenia in patients with cardiovascular and cerebrovascular diseases, thrombocytopenia caused by drug therapy, hypersplenism, thrombocytopenia during pregnancy, thrombocytopenia secondary to aplastic anemia, thrombocytopenia secondary to hypersplenism, thrombocytopenia secondary to leukemia, thrombocytopenia secondary to systemic lupus erythematosus, thrombocytopenia secondary to Sjogren's syndrome, or thrombocytopenia secondary to ionizing radiation.

Preferably, in the drug-induced thrombocytopenia, the drug is one or more of antitumor drugs, quinines, quinidines, heparins, antibiotics, and anticonvulsant drugs.

Preferably, the thrombopoiesis deficiency disease includes congenital ineffective thrombopoiesis, amegakaryocytic thrombocytopenia, Fanconi syndrome, Bernard-Soulier syndrome caused by platelet membrane glycoprotein Ib-ix deficiency or dysfunction, gray platelet syndrome, eczema-thrombocytopenia-immunodeficiency syndrome (Wiskott-Aldrich syndrome), thrombocytopenia caused by aplastic anemia and myelodysplastic syndrome, acquired ineffective thrombopoiesis, thrombopoiesis deficiency disease caused by chemotherapeutic drugs, or thrombopoiesis deficiency disease caused by radiation damage.

Preferably, the diseases associated with decrease in platelet counts include diseases caused by ineffective thrombopoiesis, diseases caused by increased platelet destruction, or thrombotic thrombocytopenic purpura.

Preferably, the diseases caused by ineffective thrombopoiesis include chronic aplastic anemia, myelodysplastic syndrome, ineffective thrombopoiesis caused by radiotherapy or ineffective thrombopoiesis caused by chemotherapy; and the diseases caused by increased platelet destruction include increased platelet destruction caused by autoimmune diseases, increased platelet destruction caused by antiphospholipid syndrome, increased platelet destruction caused by human immunodeficiency virus or increased platelet destruction caused by drug-induced thrombocytopenia.

Preferably, the drugs are tablets, capsules, granules, pills, sustained release formulations, controlled release formulations, oral solutions or patches. Preferably, the drugs comprise a pharmaceutically effective dose of the protein kinase A activator and a pharmaceutically acceptable carrier.

Preferably, the drugs are administered orally, by injection, by spray inhalation or via gastrointestinal tract.

A method of using a protein kinase A inhibitor in preparation of drugs for treating diseases associated with increase in platelet counts is provided.

Preferably, the protein kinase A inhibitor is one or more of an inorganic inhibitor and an organic inhibitor.

Preferably, the inorganic inhibitor is one or more of hydrides, oxides, acids, bases, and salts.

Preferably, the organic inhibitor is one or more of hydrocarbons, hydrocarbon derivatives, saccharides, proteins, fats, nucleic acids, and synthetic polymer materials.

Preferably, the hydrocarbons are one or more of alkenes, alkanes, alkynes, and aromatic hydrocarbons; the hydrocarbon derivatives are one or more of halogenated hydrocarbons, alcohols, phenols, aldehydes, acids, and esters; the saccharides are one or more of monosaccharides, disaccharides, oligosaccharides, and polysaccharides; the proteins are one or more of amino acids and polypeptides; and the nucleic acids are one or more of deoxyribonucleic acid and ribonucleic acid.

Preferably, the protein kinase A inhibitor is one or more of a phosphodiesterase agonist, an adenylate cyclase inhibitor, a cyclic adenosine monophosphate, an adenosine triphosphate and a substrate of protein kinase A catalytic subunit or analogs and derivatives made according to structural features thereof.

Preferably, the protein kinase A inhibitor is one or more of fasudil, N-[2-(phosphorylated bromonitroarginylamino)ethyl]-5-isoquinoline sulfonamide, C₉₄H₁₄₈N₃₂O₃₁, C₈₀H₁₃₀N₂₈O₂₄, C₂₇H₂₁N₃O₅, C₂₆H₁₉N₃O₅, C₂₀H₁₃N₃O, C₃₂H₃₁N₃O₅, C₂₂H₂₂N₄O, C₁₄H₁₇N₃O₂S.2HCl, C₁₄H₁₇N₃O₂S, C₁₁H₁₃N₃O₂S.HCl, C₁₂H₁₃ClN₂O2SHCl, C₁₂H₁₅N₅O₂S₂HCl, C₅₃H₁₀₀N₂₀O₁₂, 1-(5-quinolinesulfonyl)piperazine, 4-cyano-3-methylisoquinoline, acetamido-4-cyano-3-methylisoquinoline, 8-bromo-2-monoacyladenosine-3,5-cyclic monophosphorothioate, adenosine 3,5-cyclic monophosphorothioate, 2-O-monobutyl-cyclic adenosine monophosphate, 8-chloro-cyclic adenosine monophosphate, N-[2-(cinnamoylamino acid)]-5-isoquinolinone, reverse phase-8-hexylamino adenosine 3,5-monophosphorothioate, reverse phase-8-piperidinyladenosine-cyclic adenosine monophosphate, reverse phase-adenosine 3,5-cyclic monophosphorothioate, 5-iodotuberculin, 8-hydroxyadenosine-3,5-monophosphorothioate, calphostin C, daphnetin, reverse phase-8-chlorophenyl-cyclic adenosine monophosphate, reverse phase-cyclic adenosine monophosphate, reverse phase-8-Br-cyclic adenosine monophosphate, 9-adenylate cyclase, 1-(5-isoquinolinesulfonyl)-2-methylpiperidine, 8-hydroxyadenosine-3 ‘,5’-monophosphate, 8-hexylaminoadenosine-3′,5′-monophosphate, and reverse phase-adenosine 3′,5′-cyclic monophosphate or analogs and derivatives made according to structural features thereof.

Preferably, the diseases associated with increase in platelet counts include essential thrombocytosis diseases or secondary thrombocytosis diseases.

Preferably, the essential thrombocytosis diseases include essential thrombocytosis, chronic myelocytic leukemia, myelofibrosis and polycythemia vera, myelodysplastic syndrome or myeloproliferative neoplasm.

Preferably, the secondary thrombocytosis diseases include thrombocytosis after splenectomy, infection caused by bacteria or viruses, tumor or immune system disease.

Preferably, the drugs are tablets, capsules, granules, pills, sustained release formulations, controlled release formulations, oral solutions or patches.

Preferably, the drugs comprise a pharmaceutically effective dose of the protein kinase A inhibitor and a pharmaceutically acceptable carrier.

Preferably, the drugs are administered orally, by spray inhalation, by injection or via gastrointestinal tract.

A method of using a protein kinase A inhibitor in preparation of drugs for promoting platelet apoptosis is provided.

### Advantageous Effect

The present invention has found that PKA is in an early regulatory stage of initiating or inhibiting pathophysiological conditions to induce platelet apoptosis. PKA enhances binding to 14-3-3 by phosphorylating the serine residue at position 155 of the Bad pro-apoptotic protein, thereby promoting the release of the anti-apoptotic protein Bcl-xL to inhibit platelet apoptosis. Therefore, our results confirm that various pathophysiological factors in vitro and in vivo can induce platelet apoptosis, and PKA is at the upstream of apoptosis regulation. By increasing PKA activity, platelet apoptosis induced by storage or pathological stimulus can be significantly prevented. The technical scheme of the present invention can be specifically applied to treating diseases related to the decrease of platelet counts such as idiopathic thrombocytopenic purpura, diabetes and bacterial infection, and has a broad clinical application prospect. In addition, the protein kinase A activator of the present invention can be widely used in the storage of platelets.

The present invention investigates the effect of PKA in the regulation of platelet apoptosis by means of experimentation for the first time, and finds that PKA activity in platelets of patients with ITP, infection and diabetes decreases, and PKA regulates platelet apoptosis by means of regulating the phosphorylation of serine at BAD position 155. Inhibiting PKA activity may induce platelet apoptosis in vitro, while also reducing circulating platelet counts in the body, indicating that PKA inhibitors may participate in the treatment of thrombocytosis, and reduce platelet counts in peripheral circulating blood. PKA inhibitors have the potential to be developed into a new drug for the treatment of thrombocytosis, and is of great scientific and economic value.

### BRIEF DESCRIPTION OF THE DRAWINGS

FIGS. 1A to 1C show the test results of phosphorylated GPIbβ, GPIbβ total protein and PKA activity in platelets of patients with ITP, diabetes and sepsis.

FIG. 2 shows the test results of GPIbβ phosphorylated protein, GPIbβ total protein and PKA activity in platelets after bacterial infection.

FIG. 3 shows the test results of the percentage of platelets for platelet apoptotic mitochondrial transmembrane potential depolarization induced by protein kinase A inhibition.

FIG. 4 shows the results of western blot detection caspase-3, gelsolin protein expression and caspase-3 activity in platelets for platelet apoptosis induced by protein kinase A inhibition.

FIG. 5 shows the test results of PS exposure for platelet apoptosis induced by protein kinase A inhibition after incubation of washed platelets with different concentrations of H89.

FIG. 6 is a platelet scatter plot collected by FSC-FL1 for platelet apoptosis induced by protein kinase A inhibition.

FIG. 7 shows the results of scanning electron microscopy after different concentration gradients of protein kinase A inhibitors H89 acting on washed platelets for 160 minutes at 22° C.

FIGS. 8A to 8D show experimental results related to PKA regulation of platelet apoptosis by regulating phosphorylation of serine at BAD position 155.

FIG. 9 shows the determination results of platelets and reticulated platelets in mice counted for 0 to 8 days after injection of a PKA agonist 8-Br-cAMP (2.5 mg/mL) in male ICR mice.

FIGS. 10A to 10E show a construction process of a conditional knockout mouse and related test results.

FIGS. 11A to 11C show the results related to an increased platelet clearance ratio in PKA knockout mice.

FIGS. 12A to 12F show related results of percentages of mitochondrial transmembrane potential depolarized platelets and PS positive platelets.

FIG. 13 shows the results of platelet ΔΨm after washed platelets are incubated with the protein kinase A activator drug milrinone (8 μM), negative control, and thrombin.

FIG. 14 shows the results of platelet ΔΨm after washed platelets are incubated with the protein kinase A activator drug aminophlline (0.48 mM), negative control, and thrombin.

FIG. 15 shows the results of platelet ΔΨm after washed platelets are incubated with a protein kinase A activator drug sterilized prostaglandin E₂ solution (10 ng/ml), negative control, and thrombin.

FIG. 16 shows the results of platelet ΔΨm after washed platelets are incubated with a protein kinase A activator drug cyclic adenosine monophosphate injection (24 μg/mL), negative control, and thrombin.

FIG. 17 shows the results of platelet counts at different times after injection of the protein kinase A activator drug milrinone (1 mg/kg) (or NS) in the tail vein of mice.

FIG. 18 shows the results of platelet counts at different times after injection of the protein kinase A activator drug PGE2 (20 ng/ml) (or NS) in the tail vein of mice.

FIG. 19 shows the results of platelet counts at different times after injection of the protein kinase A activator drug cAMP (12 μg/ml) (or NS) in the tail vein of mice.

FIG. 20 shows the results of platelet counts at different times after injection of the protein kinase A activator drug aminophlline (0.24 μmol/L) (or NS) in the tail vein of mice.

FIGS. 21A to 21E show the results of experiments related to acute thrombocytopenia induced by PKA inhibition.

FIG. 22 shows the platelet ΔΨm and PS exposure test results after washed platelets are incubated with different Fasudil or negative control.

FIG. 23 shows the test results of blood sampling and counting at different times after injection of DMSO and Fasudil (1.6 μmol/L) in the control group and the experimental group after blood sampling.

### DETAILED DESCRIPTION OF THE INVENTION

1. Reagents and Materials:

The anti-GpIIb/IIIa monoclonal antibody SZ21 was provided by Prof. Ruan Changgeng, Director of Jiangsu Institute of Hematology. Dimethyl sulfoxide (DMSO) and anti-Actin primary antibody were purchased from Sigma, USA. EDTA-K2 anticoagulant tubes were purchased from Becton, Dickinson and Company, USA. Fluorescein isothiocyanate (FITC)-Annexin V was purchased from Beijing Jiamay Biotechnology Co., Ltd. FITC-goat-anti-mouse antibody was purchased from Bioworld Technology, USA. Horse radish peroxidase (HRP)-goat-anti-mouse, HRP-goat-anti-rabbit, rabbit and mouse IgG, anti-BAX, anti-BAK, anti-Bcl-xL, anti-Bcl-2, anti-Caspase-3, and anti-BAD 155 phosphorylated antibody were purchased from Santa Cruz Biotech, USA. Anti-PKA Ca antibody was purchased from CST Corporation, USA. N-[2-((p-bromocinnamyeamino)ethyl]-5-isoquinoline sulfonamide (H89), Forsklin, anti-GAPDH, anti-P53 antibody, JC-1, ECL, and PMSF were purchased from Beyotime Biotechnology Co., Ltd, China. E64 was purchased from Roche Biotech Co., Ltd., USA. A23187 was purchased from Calbiochem, USA. RNA oligonucleotides were designed and synthesized by GenePharma. Liposomal Lipofectamine™ 2000 and medium Opti-Mem I were purchased from Invitrogen Bios, USA.

2. Experimental Mice:

PKA knockout mice (B6; 129X1-Prkaca^{tmlGsm}/Mmnc) were purchased from the US MMRRC UNC in the background of C57BL/6J. All animal experiments were approved by the Ethics Committee of the First Affiliated Hospital of Suzhou University.

3. Washed Platelets:

Blood was collected from healthy adult volunteers from the median cubital vein, and the blood donors gave informed consent and signed an agreement. The experimental plan was approved by the Ethics Committee of the First Affiliated Hospital of Suzhou University and conformed to the Declaration of Helsinki.

When washed platelets were prepared, venous blood from healthy volunteers was anticoagulated with ACD (2.5% sodium citrate, 2.0% glucose, and 1.5% citric acid) at 1:7, platelet-rich plasma (PRP) was obtained by centrifugation at 1300 rpm for 20 min, the PRP was centrifuged at 1500 g for 2 min, and the supernatant was discarded. The precipitated platelets were suspended, centrifuged and washed with CGS buffer (0.123 M sodium chloride, 0.033 M glucose, 0.013 M sodium citrate, pH 6.5). The platelets were resuspended with modified Tyrode's buffer (2.5 mM zwitterionic buffer Hepes, 150 mM sodium chloride, 2.5 mM potassium chloride, 1 mM calcium chloride, 1 mM magnesium chloride, 12 mM sodium bicarbonate, 5.5 mM glucose, pH 7.4) to obtain a washed platelet suspension. The platelets were counted by a counter. The platelet suspension was adjusted to a concentration of 3×10⁸/mL, and allowed to stand at room temperature for 60 min.

4. Electron Microscope:

The washed platelets were fixed with 2.5% glutaric acid at 4° C. overnight. The washed platelets were sent to a scanning electron microscope sample room for sample preparation. Morphological analysis of the platelets was carried out by the scanning electron microscope (Japan Hitachi, S-4700). Five different fields of view were selected for observation and photographing.

5. Whole Body Irradiation and Bone Marrow Transplantation:

Male WT mice (6 weeks old) received a 9.5 Gy dose of whole body irradiation from a Co⁶⁰ source. Fetal liver cells were collected from PKA gene heterozygous pregnant mice (about 15 days pregnant). Injection was carried out according to 1 fetal mouse liver cells corresponding to one irradiated male mouse (completed within 6 h after receiving the irradiation). The male mice were put into an IVC special animal room, given with acidified water, Co⁶⁰ irradiated feed and bedding, and observed for the survival condition daily. Surviving mice after 4 weeks were tested for complete blood counts and, if the surviving mice returned to normal, the surviving mice were used in the next experiment. Whether the transplantation was successful was determined by Western Blotting to detect the expression of PKA protein in the platelets of the recipient mice.

6. Mitochondrial Membrane Potential (ΔΨm) Detection

Washed platelets (3×10⁸/mL) were incubated with different concentrations of H89 (12.5 μM, 25 μM, 37.5 μM and 50 μM) or negative control (DMSO) for 10 min at room temperature, and then platelet ΔΨm was determined using the lipophilic cationic dye JC-1. JC-1 with a final concentration of 2 μg/ml was added to the treated platelets, and the platelets were incubated at 37° C. for 20 min in the dark, and detected by using a flow cytometer. Red fluorescence indicates a mitochondrial membrane potential-dependent JC-1 polymer, and green fluorescence indicates a JC-1 monomer that does not bind to a membrane potential after depolarization of the mitochondrial membrane potential. The JC-1 monomer (λex 514 nm, λem 529 nm) and the polymer (λex 585 nm, λem 590 nm) were determined by calculating the proportion of flow red fluorescence (JC-1 polymer) or green fluorescence (JC-1 monomer).

7. PS Exposure

Washed platelets were incubated with different concentrations of H89 (12.5 μM, 25 μM, 37.5 μM and 50 μM) or negative control (DMSO) at room temperature for 10 min. After that, Annexin V buffer, H89-treated platelets, and Annexin V-FITC in a ratio of 50:10:1 were incubated for 15 min at room temperature in the dark, and detected by using a flow cytometer.

8. Platelet Shrinkage Experiment

Washed platelets were incubated with different concentrations of H89 (12.5 μM, 25 μM, 37.5 μM and 50 μM) or negative control (DMSO) at room temperature for 10 min. The platelets and the SZ21 antibody were then incubated for 30 min at room temperature. The platelets were centrifuged, resuspended with the FITC-labeled goat-anti-mouse antibody, and incubated for 30 min at room temperature in the dark. The platelets were collected by using a flow cytometer, and platelet scatter plots collected by FSC-FL1 were used to analyze the platelets. The degree of shrinkage of the platelets was evaluated by analyzing the change in FSC and evaluating the degree of FSC reduction in GPIIb/IIIa positive cells. A23187 was used as a positive control. DMSO was used as a negative control.

9. Western Blotting:

Washed platelets were incubated with different concentrations of H89 (25 μM, 50 μM, and 100 μM) or negative control (DMSO) at room temperature for 10 min. The reaction was stopped by the addition of 2× cell lysis buffer (containing 2 mM PMSF, 2 mM NaF, 2 mM Na₃VO₄, and protease inhibitor), and the platelets were lysed on ice, and sampled. The samples were tested for expression of the corresponding proteins by Western blotting.

10. RNA Interference Experiment

Double-stranded siRNA oligonucleotide of target PRKACA (positive sense: 5-GCUCCCUUCAUACCAAAGUTT-3, negative sense: 5-ACUUUGGUAUGAAGGGAGCTT-3) and negative control siRNA (positive sense: 5-UUCUCCGAACGUGUCACGUTT-3, negative sense: 5-ACGUGACACGUUCGGAGAATT-3) were designed and synthesized by GenePharma.

When hela cells were transfected, a culture plate was inoculated with 2×10⁵ hela cells one day before transfection. About 500 μL of antibiotic-free medium was added to each well to achieve a cell density of 30-50% during transfection. 1 μL/well of Lipofectamine 2000 (slightly shaken before use) was taken and diluted with 50 μL of Opti-MEM I low serum medium. The lipofectamine 2000 was slightly mixed and incubated for 5 min at room temperature. 2 μL of FAM-siRNA was taken, diluted with 50 μL of Opti-MEM I low serum medium, and slightly mixed. After being incubated for 5 min, the diluted lipofectamine 2000 was slightly mixed with the diluted FAM-siRNA, and allowed to stand for 20 min at room temperature. The FAM-siRNA-transfection reagent mixture was added to wells containing cells and a culture solution (about 400 μL), and the well plate was gently shaken to mix.

In the platelet transfection experiment, 6×10⁸/mL washed platelets were prepared aseptically and allowed to stand. 100 μL of siRNA oligonucleotide was added to 100 μL of platelets suspended in serum-free M199 medium.

The platelets were cultured in a CO₂ incubator at 37° C. for 6 h, and then the culture medium can be changed to serum-containing complete medium for culturing for 48 h. The transfection efficiency was measured by a flow cytometer after 6 h of transfection. At the end of the culture, hela cells and platelets were collected and lysed. The samples were examined for the expression degree of PKA Ca by Western blotting, and Actin was used for internal reference detection.

11. Statistical Analysis:

All data were derived from at least 3 independent experiments. Data were expressed as mean±standard error. Statistical analysis was carried out on the data using PrismVersion 5.0, and the data was subjected to unpaired T test, with p<0.05 as the differential significant cutoff value.

12. Experimental Results:

(1) PKA Activity Declines in Plasma-Incubated or Bacterially Infected Platelets in Sepsis, Diabetes, or ITP Patients

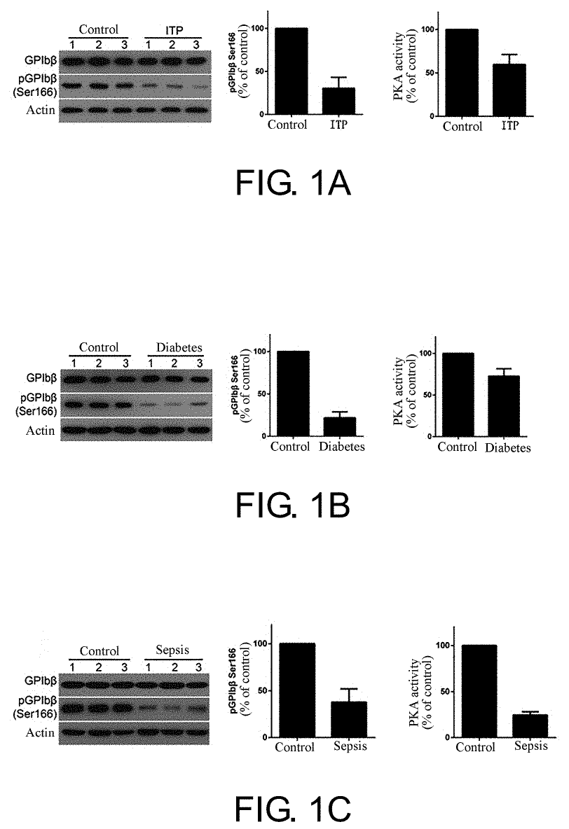

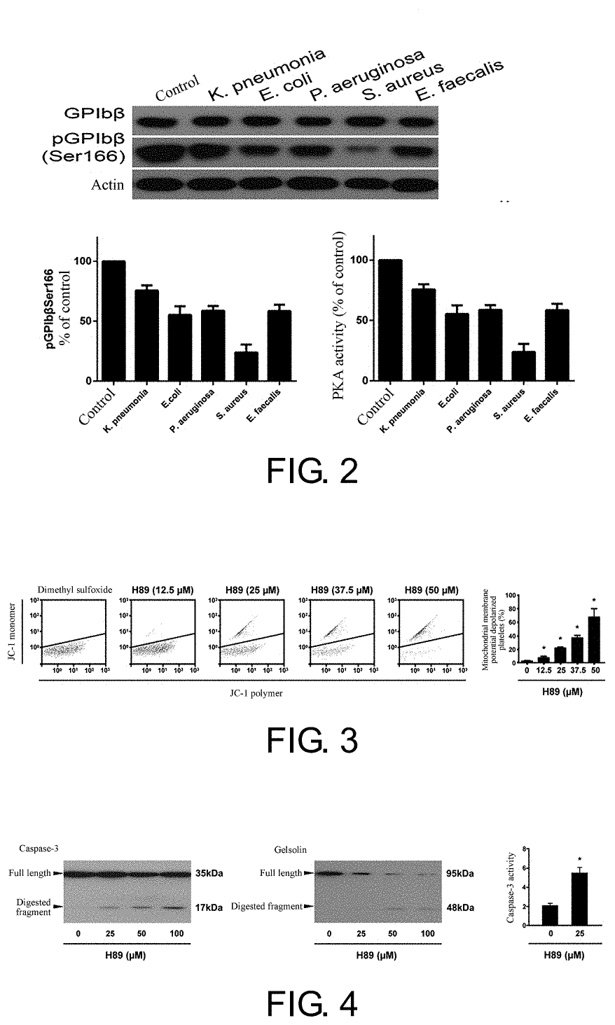

Thrombocytopenia often occurs in some high-morbidity diseases such as diabetes, ITP, sepsis or bacterial infections. We studied the role of PKA in these thrombocytopenia diseases. Platelets were collected by centrifugation from patients with ITP, diabetes, and sepsis, and platelets in healthy populations of the appropriate age and gender were used as controls. After the platelets were resuspended with MTB, the concentration was adjusted to 3×10⁸/mL. The total protein after platelet lysis was used to detect phosphorylated GPIbβ, GPIbβ total protein and PKA activity, and compared with the control group, *P is less than 0.05, and **P is less than 0.01. The MTB-diluted washed platelets (1×10⁷/mL) were co-cultured with the corresponding bacteria (diluted with MTB buffer in a ratio of 1:20) for 90 min at 37° C. At the same time, a non-bacterial culture group was set up as a negative control. The GPIbβ phosphorylated protein, GPIbβ total protein and PKA activity were detected. The results were obtained from four independent experiments of different platelet donors, and are shown in FIGS. 1A to 1C, and FIG. 2, *P is less than 0.05, and **P is less than 0.01.

We can find that normal platelet apoptosis is induced after healthy human platelets are incubated with plasma of diabetes or ITP patients, and PKA activity significantly decreases. Recent evidence suggests that E. coli and S. aureus isolated from sepsis patients can induce platelet apoptosis in vitro (Kraemer et al., 2012). Our study found that E. coli and Staphylococcus can not only induce platelet apoptosis, but also significantly reduce PKA activity. In a word, these results suggest that ITP, sepsis, and diabetic patients or bacterial infections can induce platelet apoptosis and decrease PKA activity.

(2) Inhibition of PKA Activity Induces Endogenous Pathway-Dependent Apoptosis of Platelets

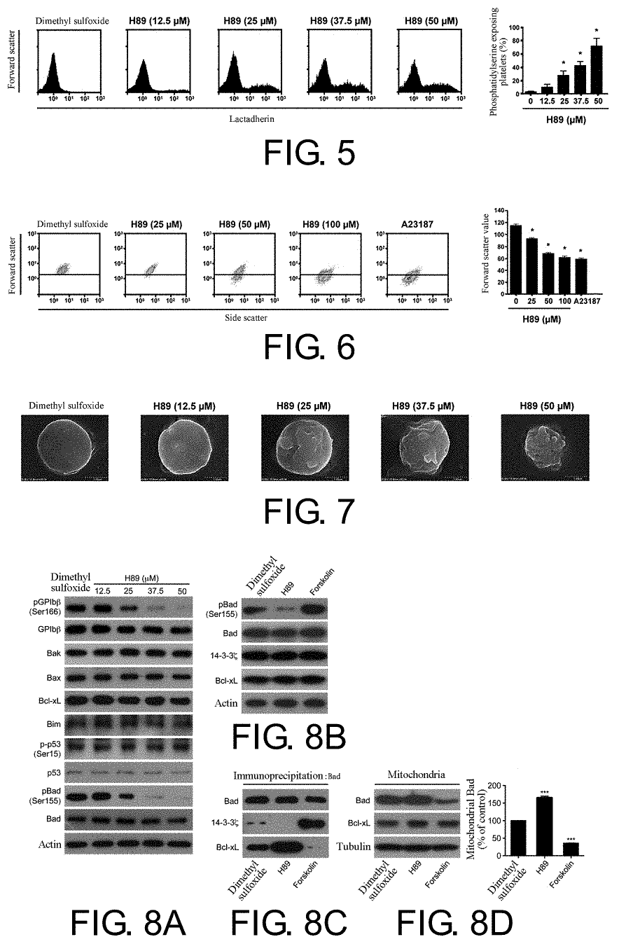

Different concentrations of H89 (0, 12.5, 25, 37.5, and 50 μM) were applied to washed platelets at 22° C. for 160 min, and mitochondrial transmembrane potential depolarization and PS exposure of platelets were detected by using the flow cytometer. The experiments were repeated four times. The results are shown in FIGS. 3 to 7. The washed platelets were pretreated with different concentrations of H89 at 22° C. for 30 min. At the same time, DMSO and A23187 treated negative control group and positive control group platelets were established. Caspase-3, gelsolin protein expression and caspase-3 activity in platelets were detected by Western blot. The FITC-labeled anti-CD41 antibody was mixed with the pre-treated platelets at a ratio of 1:10, and incubated at room temperature for 10 min in the dark. Analyzing the platelet size scatter plot, the decrease in the CD41 positive cell count represents a decrease in the platelet count. Different concentrations of H89 were applied to washed platelets at 22° C. for 160 min, while DMSO negative control platelets were established. After platelets were immobilized with 1% glutaraldehyde for 30 min, the results of scanning electron microscopy were observed. The experimental results were from three independent experiments with a scale of 1 μm. The results were expressed as mean±standard deviation, and compared with the control group, *P is less than 0.05. The results were repeated three times or more.

Next we explored the role of PKA in regulating platelet apoptosis. Platelets were incubated with the PKA inhibitor H89, and the JC-1 dye labeling changes in the platelets were detected by using the flow cytometer. It was found that H89 dose-dependently induced mitochondrial membrane potential (ΔΨm) depolarization in platelets. Moreover, H89 can also induce ΔΨm depolarization in platelets in a time-dependent manner. ΔΨm depolarization is located upstream of the caspase-3 signaling pathway. Caspase-3 is one of the executioners in the caspase family, which can lead to the disintegration and collapse of cells. Our study found that H89 dose-dependently induced activation of caspase-3 and digestion of caspase-3 substrate gelsolin. Phosphatidylserine (PS) exposure is another distinct marker molecule for endogenous pathway-dependent apoptosis. Our study found that H89 could induce PS exposure on the platelet surface in a dose-dependent manner.

During apoptosis, mitochondrial dysfunction initiates bioenergetic destruction, which ultimately leads to disruption of plasma membrane integrity and leads to morphological changes. The experimental results showed that H89 could induce GPIIb/IIIa-positive platelets to decrease in forward scatter (FSC), indicating that platelets shrank in morphology after inhibition of PKA activity. In addition, H89 dose-dependently induce platelets to cause typical apoptotic morphological changes, including cell membrane vesicles, pseudopods, shrinkage, degranulation and so on. In a word, these results indicate that PKA inhibition can induce endogenous pathway-dependent apoptosis of platelets.

(3) PKA Regulates Platelet Apoptosis by Regulating Phosphorylation of BAD Position 155

Next, we further explored the mechanism by which PKA inhibits induction of platelet apoptosis. At room temperature, 3×10⁸/mL washed platelets were incubated with different concentration gradients of H89 or the DMSO internal reference for 160 min. The platelets were lysed on ice for 30 min with an equal volume of lysis buffer. The product was electrophoretically separated by SDS-PAGE to obtain protein fragments of different sizes. The protein fragments were blocked in skim milk powder for one hour and then the primary antibody was added for incubation. Finally, ECL luminescence showed the bands of interest proteins (FIG. 8A). The washed platelets were pretreated with 37.5 uM H89, 10 uM forskin and the DMSO internal reference respectively for 160 min at room temperature. Cytoplasmic proteins (FIG. 8B) and mitochondrial proteins (FIG. 8D) were extracted from the platelets, respectively. The interest proteins were detected by western blot. The amount of the interest proteins was analyzed by Image J software. Four experiments were counted to show the results in mean±standard deviation. After lysis, the pre-treated platelets were centrifuged at 17,000 g for 10 min at 4° C. The obtained supernatant was incubated with the corresponding antibody for precipitation overnight, and after incubation with protein A/G+agarose beads at 4° C. for 2 hours, the beads were eluted for protein hybridization (FIG. 8C). Four experiments were counted to show the results in mean±standard deviation, *P is less than 0.05, and **P is less than 0.01.

As a result, it was found that the PKA inhibition dose-dependently decreased the degree of phosphorylation of serine at the platelet GPIbβ position 166, thereby demonstrating a decrease in PKA activity. However, our study found that there was no significant change in the expression levels of apoptotic execution proteins BAK and BAX and anti-apoptotic proteins Bcl-2 and Bcl-xL in apoptotic platelets. It has been reported that in S49 lymphoma cells, PKA increases the expression of Bim by regulation to avoid apoptosis of tumor cells. However, our study found that Bim protein levels did not change significantly during platelet apoptosis in which PKA participated in regulation, which ruled out the regulation of Bim.

It has been reported that PKA inhibition can promote the expression of P53, and phosphorylated P53 in platelets of diabetic patients can directly induce inactivation of anti-apoptotic protein Bcl-xL, which in turn promotes platelet apoptosis. However, no changes were detected in P53 or phosphorylated P53 in platelet apoptosis in which PKA participated.

In nucleated cells, PKA regulates apoptosis by regulating the phosphorylation of serine at the BAD position 155 and regulating the binding of the 14-3-3 protein to the anti-apoptotic protein Bcl-xL. Under the condition of dephosphorylation of BAD position 155, BAD and Bcl-xL form a dimer, releasing apoptosis executors BAK and BAX, and further leading to enhanced mitochondrial membrane permeability and apoptosis. We found that the serine at the BAD position 155 showed a decrease in the degree of phosphorylation in platelets induced by PKA inhibition. Moreover, H89 decreased and forskolin enhanced the phosphorylation of serine at the BAD position 155, which in turn affected the decrease and increase of Bcl-xL protein on the mitochondrial membrane. Most importantly, the results of co-immunoprecipitation showed that Bcl-xL and BAD were significantly reduced or enhanced under H89 or forskolin stimulation, suggesting that H89 or forskolin can regulate the activity of anti-apoptotic protein Bcl-xL by regulating the interaction between BAD and Bcl-xL. Therefore, these data indicate that PKA regulates platelet apoptosis by regulating phosphorylation of serine at the BAD position 155.

(4) PKA Activators Increase Circulating Platelet Counts

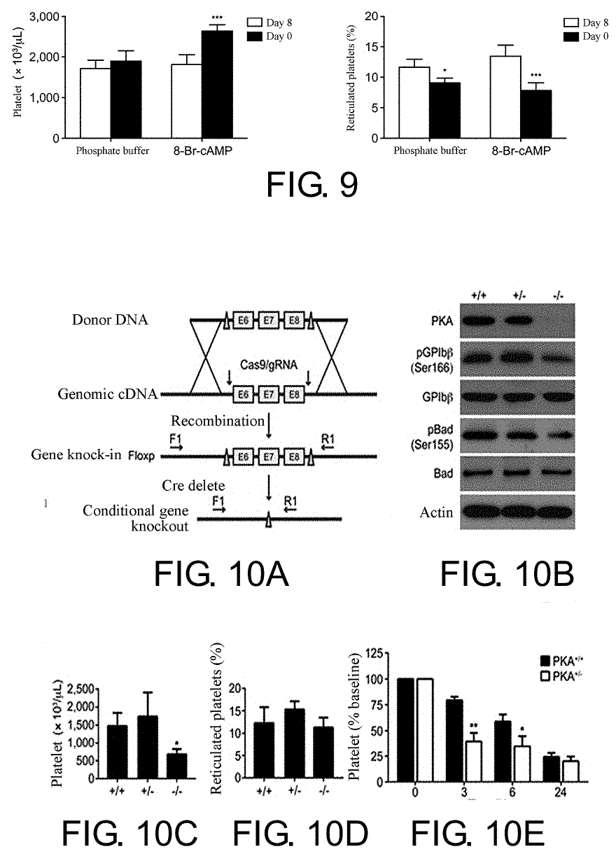

On the other hand, PKA activation can promote the phosphorylation level of serine at the BAD position 155, thereby preventing the occurrence of apoptosis. Therefore, PKA activators may prevent apoptosis of senescent platelets and prolong the lifespan of platelets. To verify this hypothesis, we injected male ICR mice with PKA agonist 8-Br-cAMP (2.5 mg/mL) via the tail vein every 24 h, and set up the PBS group as a negative control. After 8 days, the platelets and reticulated platelets in the mice were counted. 5-6 mice were used in each of the experimental group and the control group, *P is less than 0.05, and **P is less than 0.01 (FIG. 9).

As a result, it was found that the platelet counts increased day by day and peaked on the eighth day. Correspondingly, the reticulated platelets decreased day by day, indicating that the increase in platelets is not a result of increased platelet production (FIG. 9). Moreover, there was no significant change in JC-1 labeled platelets. These data indicate that increased PKA activity protects platelets from apoptosis and prolongs the platelet lifespan in vivo.

(5) The Platelet Clearance Ratio in PKA Knockout Mice Increases

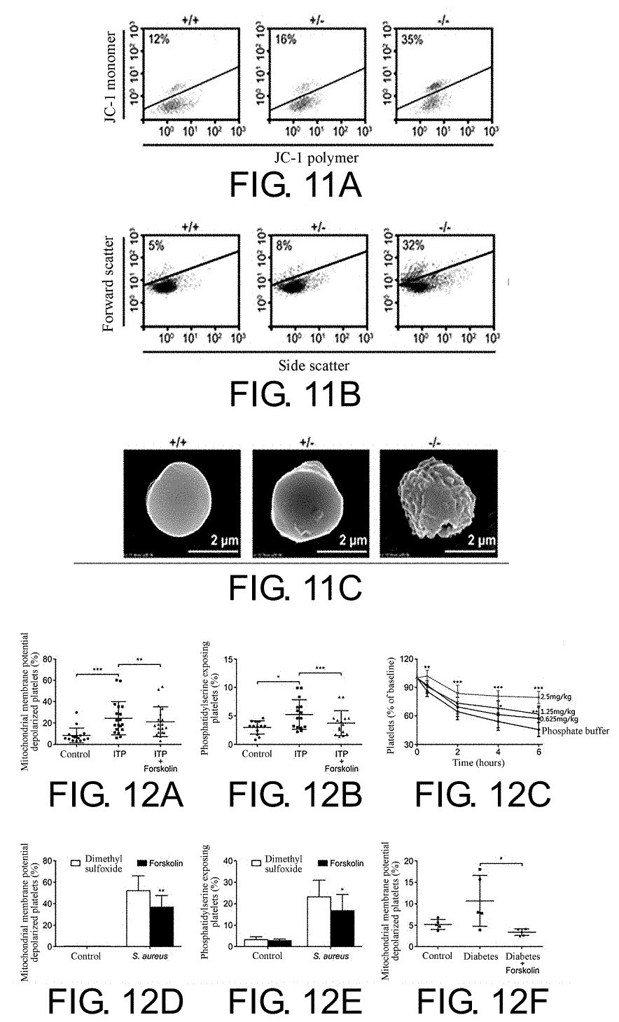

First, conditional knockout mice were constructed (FIG. 10A). The expression of PKA, Bad, GBIbβ, phosphorylated Bad Ser-155 and phosphorylated GBIbβ Ser-166 in platelets was detected by Western blot, and at least 5 mice were used in each experimental group (FIG. 10B). Platelets were counted by a Sysmex XP-100 blood analyzer, and 7 WT mice, 7 PKA+/− mice, and 5 PKA−/− mice were statistically analyzed (FIG. 10C). Whole blood was labeled with thiazole orange (0.5 μg/mL) and anti-CD41 (20 μg/mL) antibody, and incubated at room temperature for 15 min, and the reticulated platelet count was measured by using the flow cytometer. Data were obtained from the 7 WT mice, the 7 PKA+/− mice, and the 5 PKA−/− mice (FIG. 10D). Anti-platelet antibody R300 (0.15 μg/kg) was intraperitoneally injected into the WT and PKA+/− mice, and blood was collected from the orbit. Platelets were counted by a Sysmex XP-100 blood analyzer, and 6 WT mice and 6 PKA+/− mice were respectively statistically analyzed (FIG. 10E). The washed platelets were incubated with JC-1 (2 μg/mL) for 10 min in the dark, and the mitochondrial transmembrane potential depolarization level was detected by using the flow cytometer (FIG. 11A). FITC-labeled anti-CD41 antibody was mixed with the platelets in a ratio of 1:10, gently mixed and incubated for 10 min at room temperature (FIG. 11B). Analyzing the platelet size scatter plots, a decrease in the CD41 positive cell counts represents a decrease in the platelet counts. After the washed platelets were immobilized with 1% glutaraldehyde for 30 min, the results were observed by scanning electron microscopy, and the scale was 2 μm (FIG. 11C). *P is less than 0.05, **P is less than 0.01, the experimental results were from three independent experiments, and the figure represents at least 5 mice per genotype.

Most PKA Ca knockout mice die during the perinatal period, so we established a bone marrow transplantation method for transplanting liver cells from PKA Ca knockout mouse fetuses to irradiated wild mice. The genotype of the transplanted fetal mouse liver was identified by PCR assay. Stable transplantation results were obtained after 4 months of transplantation in mice, and platelets with PKA deficiency after transplantation were verified by Western blot. There were no significant differences between the transplanted PKA−/−, PKA+/−, and WT mice in red blood cells, white blood cell count, and hemoglobin concentration. The platelet count was significantly reduced in PKA+/− mice compared with WT mice. There was no difference in reticulated platelets in PKA+/− and WT, suggesting that the reduction in platelet count was not due to decrease in platelet production, but to acceleration of platelet clearance. Moreover, we found that injection of the anti-platelet antibody R300 could induce platelet apoptosis and meanwhile could rapidly induce platelet clearance in PKA+/− faster than in WT. Interestingly, platelets in PKA−/− mice significantly decreased, and platelets in PKA−/− mice exhibited typical apoptotic changes.

To avoid possible interference of bone marrow transplantation in PKA Ca knockout mice, we constructed PKA Ca conditional knockout mice. The PKA Ca conditional knockout mice co-produced with PF4 Cre mice to obtain PKA conditional knockout mice in platelets. Conditional knockout C-PKA−/−, C-PKA+/−, and C-PKA+/+ mice did not show significant differences in red blood cells, white blood cell counts, and hemoglobin concentration changes. Moreover, PKA+/− mice did not have any tendency to self-bleed or thrombus compared to RIP3−/− mice. PKA activity in heterozygous and homozygous mice showed a dose-dependent change. Different types of mice had no obvious abnormalities in the circulating platelet counts. However, PS exposure significantly increased in PKA knockout mice. Tracking biotinylated platelets in vivo found that PKA knockout shortened the platelet lifespan in a dose-dependent manner. To confirm that the shortening of the platelet lifespan is caused by intrinsic factors of platelets, we transplanted platelets from PKA knockout mice into wild-type mice. Platelets in PKA−/− and PKA+/− mice clearly showed a shortened lifespan compared to platelets in WT mice.

(6) BAD Phosphorylation Decrease Occurs in PKA Knockout Mice

Then, we explored the mechanism of platelet apoptosis in PKA knockout mice. Consistent with the results of our previous human platelet in vitro experiments, the expression of P53 increased in a dose gradient manner in heterozygous and homozygous mice, while PKA catalytic subunit expression and PKA activity significantly decreased. Moreover, the phosphorylation of serine at the BAD position 155 of platelets in PKA knockout mice decreased. However, PKA Ca knockout platelets showed no significant changes in BAK, BAD, BAX, and Bcl-xL compared to wild-type platelets. In a word, these mouse results validate our findings in human platelets, demonstrating that inhibition of PKA results in the occurrence of platelet apoptosis mediated by the phosphorylation of serine at the BAD position 155.

(7) Increase of PKA Activity to Protect Platelets from Apoptosis and Clearance Induced by Pathological Conditions

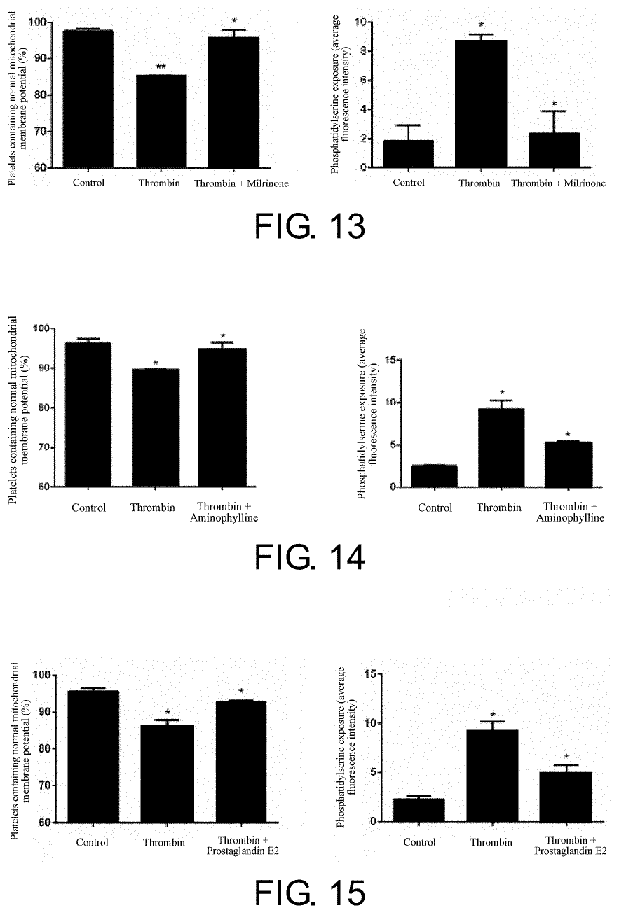

Washed platelets were pretreated with 5 uM forskin and DMSO at 22° C. for 5 min, and then co-cultured with serum of an ITP patient for 12 h at room temperature. At the same time, healthy adult serum was set up as a control. The percentage of mitochondrial transmembrane potential depolarized platelets (FIG. 12F) was detected by using the flow cytometer. ICR mice were given intravenous injection of a single dose of 8-Br-cAMP (0.0625, 1.25, 2.5 mg/kg) and the internal reference, followed by intraperitoneal injection of the Fc inhibitor to remove Fc-mediated platelet clearance. After 10 min, the anti-platelet antibody R300 (0.2 μg/kg) was intraperitoneally injected into the mice, and the whole blood was collected from the orbital vein at different time points. The platelets were counted by a Sysmex XP-100 blood analyzer, 6 mice were used per group, and the results were expressed as mean±standard deviation (FIG. 12C). Washed platelets were pretreated with 5 uM forskin and DMSO at 22° C. for 5 min, and then co-cultured with a Staphylococcus aureus suspension for 90 min at room temperature. At the same time, a negative platelet control group not treated with S. aureus liquid was set. The percentage of mitochondrial transmembrane potential depolarized platelets (FIG. 12D) and PS positive platelets (FIG. 12E) were detected by using the flow cytometer. The experiment was repeated three or more times and the results were expressed as mean±standard deviation. Washed platelets were pretreated with 5 uM forskin and DMSO at 22° C. for 5 min, and then co-cultured with serum of a diabetic patient for 12 h at room temperature. At the same time, healthy adult serum was set up as a control. The percentage of mitochondrial transmembrane potential depolarized platelets (FIG. 12A) and PS positive platelets (FIG. 12B) were detected by using the flow cytometer.

Platelet apoptosis appears to be a major cause of dysfunction and rapid clearance of stored platelets. To determine the role of PKA in platelet storage damage, PKA activators or inhibitors were added during platelet storage. As expected, PKA inhibitors were the first to initiate platelet apoptosis. Interestingly, the PKA activator Forskolin significantly delayed the onset of platelet apoptosis. It is well known that intrinsic programmed apoptosis of mitochondrial membrane potential depolarization regulation is an irreversible process. These data not only further validate the role of PKA in regulating platelet apoptosis, but also indicate that PKA is located upstream of mitochondrial depolarization-regulated apoptosis.

In addition to apoptosis, there are other changes in storage damage that result in the clearance of stored platelets in the body. Therefore, we investigated whether PKA activation protects platelet from apoptosis while preventing the stored platelets from being cleared. It was found that Forsklin clearly protects the stored platelets and inhibits their clearance by the body. On the other hand, H89 speeds up the clearance. These data suggest that PKA-regulated apoptosis plays a key role in platelet storage damage, and suggest a new method to effectively prolong the lifespan of stored platelets in blood banks.

Autoantibodies in patients with ITP, especially refractory ITP, induce platelet apoptosis and initiate platelet destruction. Consistent with previous reports, our study found that antiplatelet serum in ITP patients significantly induced platelet apoptosis. However, platelets pre-incubated with Forskolin significantly reduced serum-induced platelet apoptosis. To clarify the role of PKA activators in platelet clearance in vivo, we established an ITP mouse model using the anti-platelet monoclonal mixed antibody R300. Co-incubation of R300 with platelets in vitro induces platelet apoptosis. Injection of an Fc receptor blocker into mice can block Fc-dependent platelet destruction. PKA activators can inhibit antiplatelet antibody-induced platelet clearance in a dose-dependent manner. These results not only demonstrate the role of PKA in protecting antibody-induced platelet apoptosis and clearance, but also suggest new strategies for the treatment of ITP.

Then, we found that PKA activation is effective in preventing apoptosis induced by incubation of platelets and S. aureus isolates from sepsis patients as well as plasma in diabetic patients. In summary, these data indicate that PKA is an early regulatory protein for platelet apoptosis. Most importantly, the results of this study are of great significance for the treatment of thrombocytopenia induced by different pathophysiological stimuli and for controlling the lifespan of platelets in vivo.

(8) Experiments and Results of Inhibition of Platelet Apoptosis by the Same Type of Drugs Mitochondrial Membrane Potential Detection:

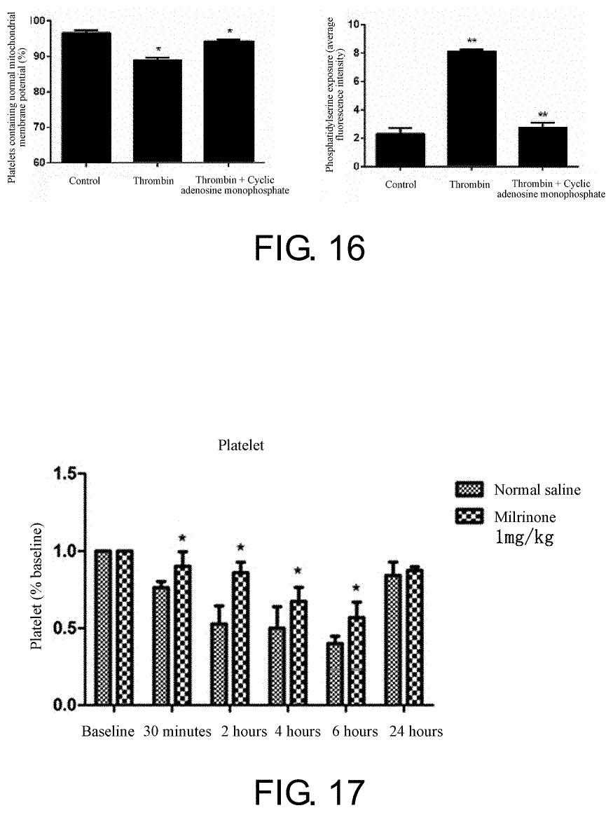

Washed platelets (3×10⁸/mL) were incubated with different PKA agonists (aminophylline 0.48 mM, sterilized prostaglandin E₂ solution 10 ng/ml, milrinone 8 μM, and cyclic adenosine monophosphate injection 24 μg/mL) or negative control (normal saline) for 10 min at room temperature. Thereafter, except the negative control, thrombin (0.1 U/ml) was added to each group, and incubation was carried out for 30 min at 37° C. Platelet ΔΨm was measured using the lipophilic cationic dye JC-1. JC-1 with a final concentration of 2 μg/ml was added to the treated platelets, and the platelets were incubated at 37° C. for 5 min in the dark, and detected by using the flow cytometer. Red fluorescence indicates a mitochondrial membrane potential-dependent JC-1 polymer, and green fluorescence indicates a JC-1 monomer that does not bind to a membrane potential after depolarization of the mitochondrial membrane potential. The JC-1 monomer (λex 514 nm, λem 529 nm) and the polymer (λex 585 nm, λem 590 nm) were determined by calculating the proportion of flow red fluorescence (JC-1 polymer) or green fluorescence (JC-1 monomer) (FIG. 13 to FIG. 16).

PS Exposure:

Washed platelets (3×10⁸/mL) were incubated with different PKA agonists (aminophylline 0.48 mM, sterilized prostaglandin E₂ solution 10 ng/ml, milrinone 8 μM, and cyclic adenosine monophosphate injection 24 μg/mL) or negative control (normal saline) for 10 min at room temperature. Thereafter, except the negative control, thrombin (0.1 U/ml) was added to each group, and incubation was carried out for 30 min at 37° C. After that, Annexin V buffer, treated platelets, and Annexin V-FITC in a ratio of 50:10:1 were incubated for 15 min at room temperature in the dark, and detected by using the flow cytometer (FIG. 13 to FIG. 16).

Milrinone can Inhibit Platelet Clearance In Vivo

12 ICR mice were divided into two groups with 6 mice in each group. The 6 mice in the experimental group were injected with milrinone (1 mg/kg), and the 6 mice in the control group were injected with normal saline (NS). 10 min after the mice were injected with the milrinone (1 mg/kg) (or NS) in the tail vein, the mice were intraperitoneally injected with the R300 antibody (0.1 mg/kg). Blood sampling and counting were then carried out at each time point. From the results, we can see that 1 mg/kg milrinone significantly increased the platelet counts in peripheral blood of mice (FIG. 17).

PGE2 can Inhibit Platelet Clearance In Vivo

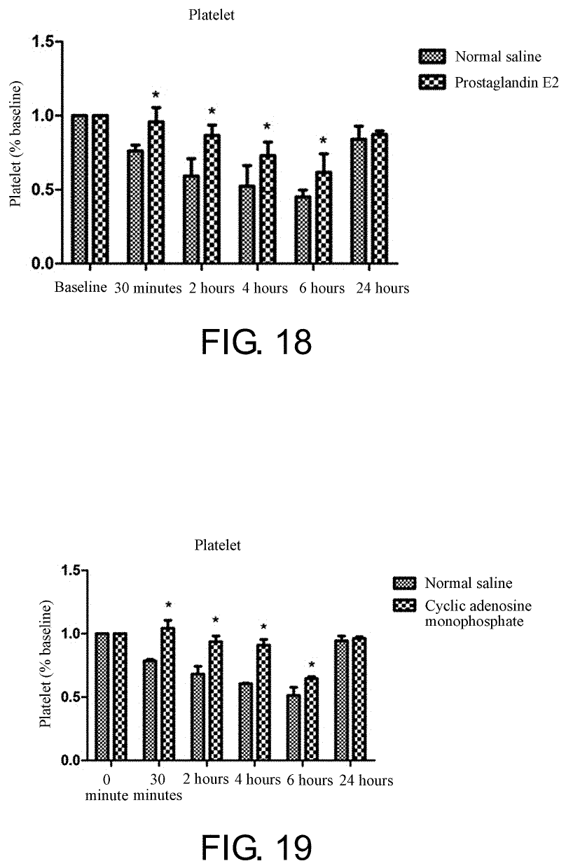

First, the mice were sampled for blood as a reference value. Then the control group and the test group were injected with 0.9% NS and PGE2 (20 ng/ml), respectively. After 10 min, R300 (0.1 μg/g) was injected. Blood sampling and counting were then carried out at 30 min, 2 h, 4 h, 6 h, and 24 h. At 30 min, the platelet counts of the NS group and the PGE2 group were P<0.05, which has statistic difference (FIG. 18).

cAMP can Inhibit Platelet Clearance In Vivo

First, the mice were sampled for blood as a reference value. Then the control group and the test group were injected with 0.9% NS and cAMP (12 μg/ml), respectively. After 10 min, R300 (0.1 μg/g) was injected. Blood sampling and counting were then carried out at 30 min, 2 h, 4 h, 6 h, and 24 h. At 30 min, the platelet counts of the NS group and the cAMP group were P<0.05, which has statistic difference (FIG. 19).

Aminophyline can Inhibit Platelet Clearance In Vivo

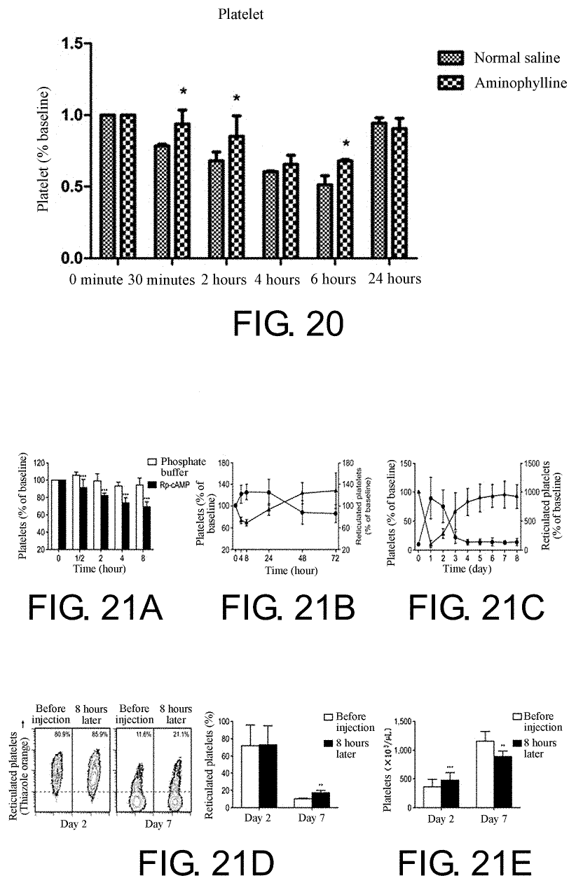

First, the mice were sampled for blood as a reference value. Then the control group and the test group were injected with 0.9% NS and aminophylline (0.24 mmol/L), respectively. After 10 min, R300 (0.1 μg/g) was injected. Then blood sampling and counting were carried out at 30 min, 2 h, 4 h, 6 h, and 24 h (FIG. 20).

(9) PKA Inhibition Induces Acute Thrombocytopenia In Vivo

Next we further explored the role of PKA in platelet lifespan in vivo. Male ICR mice were injected with a single dose of Rp-cAMPS (50 mg/kg) via the tail vein, and the platelet counts and reticulated platelet counts in mice are detected at different time points. The male ICR mice were injected intraperitoneally with a single dose of anti-platelet antibody R300 according to 0.15 mg/kg. Clearing platelets could cause severe thrombocytopenia. Reticulated platelets increased and newly synthesized platelets were released into the peripheral circulation. After about 3 days, the platelet counts in the body returned to normal. The male ICR mice were injected intraperitoneally with a single dose of anti-platelet antibody R300 according to 0.15 mg/kg. Rp-cAMPS (50 mg/kg) was injected into the tail vein after 2 days and 7 days. Platelets and reticulated platelets were counted before injection and 8 hours after injection of Rp-cAMPS, respectively. Male ICR mice were injected with PKA agonist 8-Br-cAMP (2.5 mg/mL) via the tail vein every 24 hours, and the PBS group was set up as a negative control. Platelets and reticulated platelets were counted in mice 8 days later, 5 to 6 mice were established in each of the experimental group and the control group, *P is less than 0.05, and **P is less than 0.01 (FIGS. 21A to 21E).

The PKA inhibitor reverse phase-cyclic adenosine monophosphate (Rp-cAM7PS) (non-reagent control) was injected into ICR mice via the tail vein. It was found that the platelet count decreased by 30% of the normal platelet count when tested after 2 hours. Tested after 8 hours, the platelet count dropped to the lowest value. Moreover, after Rp-cAMPS injection, platelets showed depolarization of ΔΨm, indicating that platelets undergo apoptosis.

Apoptosis occurs in senescent or stored platelets, and PKA activity decreases accordingly. Consistent with these results, we found that injection of Rp-cAMPS into mice induced a decrease in platelet counts in vivo while promoting an increase in the reticulated platelet counts, suggesting that young platelets are resistant to Rp-cAMPS-induced apoptosis, and also suggesting that PKA inhibitors in the body are more likely to induce apoptosis in senescent platelets. In order to verify this possibility, we injected anti-platelet monoclonal mixed antibody R300 into mice by intraperitoneal injection to promote platelet clearance in mice, and artificially synchronized the platelet production rate. As expected, circulating platelets were not detected in mice 6 hours after injection, and the platelet counts returned to normal in 7 days. During this period, the proportion of reticulated platelets changed significantly. Injection of Rp-cAMPS could destruct 70% of circulating platelets in mice after 7 days of R300 antibody injection, whereas only 30% of the platelets were destructed the next day. These results confirm that senescent platelets are more susceptible to stimulation of PKA inhibitors, which in turn induces platelet apoptosis and clearance. Moreover, reducing PKA activity in platelets shortens the lifespan of circulating platelets.

(10) Fasudil can Promote Platelet Apoptosis

Mitochondrial Membrane Potential Detection:

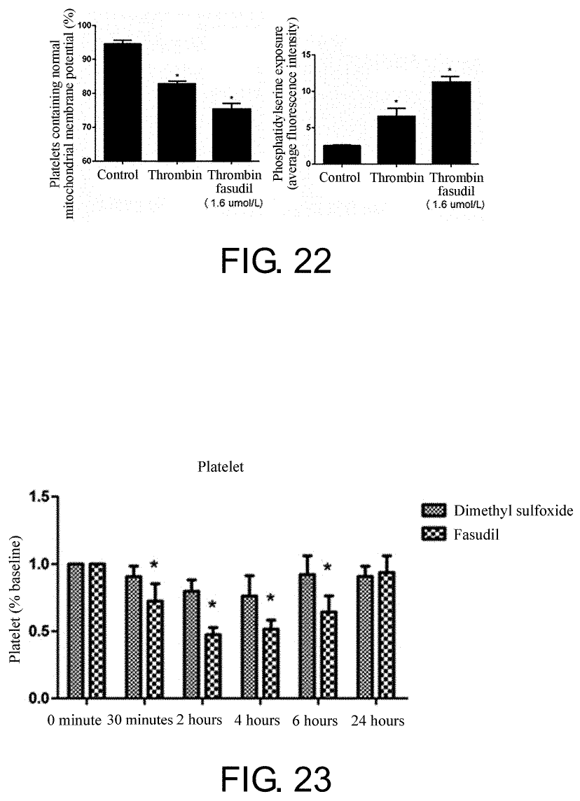

Washed platelets (3×10⁸/mL) were incubated with different Fasudil or negative control (normal saline) for 10 min at room temperature. Thereafter, except the negative control, thrombin (0.1 U/ml) was added to each group, and incubation was carried out for 30 min at 37° C. Platelet ΔΨm was measured using the lipophilic cationic dye JC-1. JC-1 with a final concentration of 2 μg/ml was added to the treated platelets, and the platelets were incubated at 37° C. for 5 min in the dark, and detected by using the flow cytometer. Red fluorescence indicates a mitochondrial membrane potential-dependent JC-1 polymer, and green fluorescence indicates a JC-1 monomer that does not bind to a membrane potential after depolarization of the mitochondrial membrane potential. The JC-1 monomer (λex 514 nm, λem 529 nm) and the polymer (λex 585 nm, λem 590 nm) were determined by calculating the proportion of flow red fluorescence (JC-1 polymer) or green fluorescence (JC-1 monomer) (FIG. 22).

PS Exposure:

Washed platelets (3×10⁸/mL) were incubated with different Fasudil or negative control (normal saline) for 10 min at room temperature. Thereafter, except the negative control, thrombin (0.1 U/ml) was added to each group, and incubation was carried out for 30 min at 37° C. After that, Annexin V buffer, treated platelets, and Annexin V-FITC in a ratio of 50:10:1 were incubated for 15 min at room temperature in the dark, and detected by using the flow cytometer (FIG. 22).

First, blood was collected from mice as a baseline value. Then the control group and the experimental group were injected with DMSO and Fasudil (1.6 μmol/L) respectively, and then blood collection and counting were carried out at 30 min, 2 h, 4 h, 6 h, and 24 h (FIG. 23).

In brief, these results indicate that PKA determines platelet lifespan and survival by regulating apoptosis. PKA inhibitors may participate in the treatment of thrombocytosis, and reduce platelet counts in peripheral circulating blood. Our research provides new ideas for the clinical treatment of thrombocytosis. Inhibition of PKA activity may become a new means of clinical treatment of thrombocytosis. PKA inhibitors have the potential to be developed into a new drug for the treatment of thrombocytosis, and are of great scientific and economic value.

What is claimed is:

1. A method of using a protein kinase A activator in preparation of drugs for treating diseases associated with decrease in platelet counts, wherein the protein kinase A activator is one or more of a phosphodiesterase inhibitor, an adenylate cyclase agonist, a cyclic adenosine monophosphate and a substrate of protein kinase A catalytic subunit, or analogs and derivatives made according to structural features thereof.

2. The method of using the protein kinase A activator in preparation of drugs for treating diseases associated with decrease in platelet counts according to claim 1, wherein the protein kinase A activator is adenosine triphosphate or analogs and derivatives made according to structural features thereof.

3. The method of using the protein kinase A activator in preparation of drugs for treating diseases associated with decrease in platelet counts according to claim 1, wherein the diseases associated with decrease in platelet counts comprise immune thrombocytopenia, infection-induced thrombocytopenia, secondary thrombocytopenia, drug-induced thrombocytopenia, thrombopoiesis deficiency disease, or non-immune thrombocytopenia, and the immune thrombocytopenia comprises idiopathic thrombocytopenic purpura.

4. The method of using the protein kinase A activator in preparation of drugs for treating diseases associated with decrease in platelet counts according to claim 3, wherein the infection-induced thrombocytopenia comprises bacterial infection-induced thrombocytopenia or viral infection-induced thrombocytopenia.

5. The method of using the protein kinase A activator in preparation of drugs for treating diseases associated with decrease in platelet counts according to claim 3, wherein the secondary thrombocytopenia comprises thrombocytopenia in diabetic patients, thrombocytopenia in tumor patients, thrombocytopenia in patients with cardiovascular and cerebrovascular diseases, thrombocytopenia caused by drug therapy, hypersplenism, thrombocytopenia during pregnancy, thrombocytopenia secondary to aplastic anemia, thrombocytopenia secondary to hypersplenism, thrombocytopenia secondary to leukemia, thrombocytopenia secondary to systemic lupus erythematosus, thrombocytopenia secondary to Sjogren's syndrome, or thrombocytopenia secondary to ionizing radiation, and in the drug-induced thrombocytopenia, the drug is one or more of antitumor drugs, quinines, quinidines, heparins, antibiotics, and anticonvulsant drugs.

6. The method of using the protein kinase A activator in preparation of drugs for treating diseases associated with decrease in platelet counts according to claim 3, wherein the thrombopoiesis deficiency disease comprises congenital ineffective thrombopoiesis, amegakaryocytic thrombocytopenia, Fanconi syndrome, Bernard-Soulier syndrome caused by platelet membrane glycoprotein Ib-IX deficiency or dysfunction, gray platelet syndrome, eczema-thrombocytopenia-immunodeficiency syndrome, thrombocytopenia caused by aplastic anemia and myelodysplastic syndrome, acquired ineffective thrombopoiesis, thrombopoiesis deficiency disease caused by chemotherapeutic drugs, or thrombopoiesis deficiency disease caused by radiation damage.

7. The method of using the protein kinase A activator in preparation of drugs for treating diseases associated with decrease in platelet counts according to claim 1, wherein the diseases associated with decrease in platelet counts comprise diseases caused by ineffective thrombopoiesis, diseases caused by increased platelet destruction, or thrombotic thrombocytopenic purpura, the diseases caused by ineffective thrombopoiesis comprise chronic aplastic anemia, myelodysplastic syndrome, ineffective thrombopoiesis caused by radiotherapy or ineffective thrombopoiesis caused by chemotherapy; and the diseases caused by increased platelet destruction comprise increased platelet destruction caused by autoimmune diseases, increased platelet destruction caused by antiphospholipid syndrome, increased platelet destruction caused by human immunodeficiency virus or increased platelet destruction caused by drug-induced thrombocytopenia.

8. The method of using the protein kinase A activator in preparation of drugs for treating diseases associated with decrease in platelet counts according to claim 1, wherein the drugs are tablets, capsules, granules, pills, sustained release formulations, controlled release formulations, oral solutions or patches.

9. The method of using the protein kinase A activator in preparation of drugs for treating diseases associated with decrease in platelet counts according to claim 1, wherein the drugs comprise a pharmaceutically effective dose of the protein kinase A activator and a pharmaceutically acceptable carrier.

10. The method of using the protein kinase A activator in preparation of drugs for treating diseases associated with decrease in platelet counts according to claim 1, wherein the drugs are administered orally, by injection, by spray inhalation, or via gastrointestinal tract.

11. A method of using a protein kinase A activator in preparation of drugs for treating diseases associated with decrease in platelet counts, wherein the protein kinase A activator is one or more of drugs amrinone, milrinone, enoximone, aminophylline, dinoprostone, iloprost, cilostazol, cilostamide, and dipyridamole or analogs and derivatives made according to structural features thereof, or one or more of Ginkgo biloba extract, quercetin, meglumine adenosine cyclophosphate, cyclic adenosine monophosphate, forskolin, 8-bromoadenosine-3′,5′-cyclic monophosphate, 8-bromo-cyclic adenosine monophosphate, 8-piperidinyladenosine-cyclic adenosine monophosphate, 8-chloro-cyclic adenosine monophosphate, adenosine 3,5-cyclic monophosphate, N6-benzoyl-cyclic adenosine monophosphate, (S)-adenosine, cyclic 3′,5′-(hydrogen phosphorothioate)triethyl, 3-isobutyl-1-methylxanthine, 8-chlorophenyl- cyclic adenosine monophosphate, adenosine 3,5-cyclic monophosphate, adenosine 3,5-cyclic monophosphorothioate, 8-bromo-cyclic adenosine monophosphate, specific 5,6-4,5-dicyanoimidazole-cyclic adenosine monophosphate, specific 8-chlorophenyl-cyclic guanosine monophosphate sodium salt, specific adenosine 3′,5′-cyclic monophosphorothioate triethyl salt, specific cyclic adenosine monophosphate, dibutyryl-cyclic adenosine monophosphate, N6-monoacyladenosine 3′,5′-cyclic monophosphate, 8-bromoadenosine 3′,5′-cyclic monophosphorothioate, 8-bromoadenosine 3′,5′-cyclic monophosphate, N6-benzoyl-cyclic adenosine monophosphate, and erythro-9-amino-β-hexyl-α-methyl-9H-purine-9-ethanol hydrochloride-9-adenine hydrochloride or analogs and derivatives made according to structural features thereof.

12. A method of using a protein kinase A inhibitor in preparation of drugs for treating diseases associated with increase in platelet counts, wherein the protein kinase A inhibitor is one or more of a phosphodiesterase agonist, an adenylate cyclase inhibitor, a cyclic adenosine monophosphate, an adenosine triphosphate and a substrate of protein kinase A catalytic subunit or analogs and derivatives made according to structural features thereof, or one or more of fasudil, N-[2-(phosphorylated bromonitroarginylamino)ethyl]-5-isoquinoline sulfonamide, C₉₄H₁₄₈N₃₂O₃₁, C₈₀H₁₃₀N₂₈O₂₄, C₂₇H₂₁N₃O₅, C₂₆H₁₉N₃O₅, C₂₀H₁₃N₃O, C₃₂H₃₁N₃O₅, C₂₂H₂₂N₄O, C₁₄H₁₇N₃O₂S.2HCl, Cl₄H₁₇N₃O₂S, C₁₁H₁₃N₃O₂S.HCl, C₁₂H₁₃C₁N₂O₂SHCl, C₁₂H₁₅N₅O₂S₂HCl, C₅₃H₁₀₀N₂₀O₁₂, 1-(5-quinolinesulfonyl)piperazine, 4-cyano-3-methylisoquinoline, acetamido-4-cyano-3-methylisoquinoline, 8-bromo-2-monoacyladenosine-3,5-cyclic monophosphorothioate, adenosine 3,5-cyclic monophosphorothioate, 2-O-monobutyl-cyclic adenosine monophosphate, 8-chloro-cyclic adenosine monophosphate, N-[2-(cinnamoylamino acid)]-5-isoquinolinone, reverse phase-8-hexylamino adenosine 3,5-monophosphorothioate, reverse phase-8-piperidinyladenosine-cyclic adenosine monophosphate, reverse phase-adenosine 3,5-cyclic monophosphorothioate, 5-iodotuberculin, 8-hydroxyadenosine-3,5-monophosphorothioate, calphostin C, daphnetin, reverse phase-8-chlorophenyl-cyclic adenosine monophosphate, reverse phase-cyclic adenosine monophosphate, reverse phase-8-Br-cyclic adenosine monophosphate, 9-adenylate cyclase, 1-(5-isoquinolinesulfonyl)-2-methylpiperidine, 8-hydroxyadenosine-3′,5′-monophosphate, 8-hexylaminoadenosine-3′,5′-monophosphate, and reverse phase-adenosine 3′,5′-cyclic monophosphate or analogs and derivatives made according to structural features thereof.

13. The method of using the protein kinase A inhibitor in preparation of drugs for treating diseases associated with increase in platelet counts according to claim 12, wherein the diseases associated with increase in platelet counts comprise essential thrombocytosis diseases or secondary thrombocytosis diseases, and the essential thrombocytosis diseases comprise essential thrombocytosis, chronic myelocytic leukemia, myelofibrosis and polycythemia vera, myelodysplastic syndrome or myeloproliferative neoplasm, and the secondary thrombocytosis diseases comprise thrombocytosis after splenectomy, infections caused by bacteria or viruses, tumors or immune system diseases.

14. The method of using the protein kinase A inhibitor in preparation of drugs for treating diseases associated with increase in platelet counts according to claim 12, wherein the drugs are tablets, capsules, granules, pills, sustained release formulations, controlled release formulations, oral solutions or patches.

15. The method of using the protein kinase A inhibitor in preparation of drugs for treating diseases associated with increase in platelet counts according to claim 12, wherein the drugs comprise a pharmaceutically effective dose of the protein kinase A inhibitor and a pharmaceutically acceptable carrier.

16. The method of using the protein kinase A inhibitor in preparation of drugs for treating diseases associated with increase in platelet counts according to claim 12, wherein the drugs are administered orally, by spray inhalation, by injection or via gastrointestinal tract.

17. A method of using a protein kinase A inhibitor in preparation of drugs, comprising the use of the protein kinase A inhibitor in preparation of the drugs for promoting platelet apoptosis.
